(19) United States
(12) Patent Application Publication
(10) Pub. No.: US 2024/0165149 A1
(43) Pub. Date: May 23, 2024

(54) NUTRACEUTICAL COMPOSITION COMPRISING INULIN FOR TREATING AND PREVENTING OCULAR DISORDERS OR INTESTINAL MICROBIOTA IMBALANCE

(71) Applicant: OPHTALMIS, Monaco (MC)

(72) Inventors: Martine CLARET, Saint Sulpice (CH); Claude CLARET, Saint Sulpice (CH); Caroline CHATARD-BAPTISTE, Nice (FR); Amir BONO, Nice (FR)

(21) Appl. No.: 18/551,781

(22) PCT Filed: Mar. 22, 2022
(86) PCT No.: PCT/EP2022/057466

§ 371 Date: Sep. 21, 2023

(30) Foreign Application Priority Data
Mar. 22, 2021 (FR) FR2102836

(51) Int. Cl. A61K 31/733; A61K 31/205; A61K 31/37; A61K 31/525; A61K 31/593; A61K 33/30; A61K 36/73; A61P 1/00

(52) U.S. Cl. CPC A61K 31/733; A61K 31/205; A61K 31/37; A61K 31/525; A61K 31/593; A61K 33/30; A61K 36/73; A61P 1/00

(57) ABSTRACT

The present invention relates to a nutraceutical composition comprising inulin and a Chilean wineberry extract, and also other ingredients such as amino acids, vitamins and/or trace elements, in particular for the use thereof in preventing and/or treating ocular pathological conditions and/or in preventing and/or treating intestinal microbiota imbalances.

### FIELD OF THE INVENTION

The present invention relates to a nutraceutical composition comprising inulin and a Chilean wineberry extract, and also other ingredients such as amino acids, vitamins and/or trace elements, in particular for the use thereof in preventing and/or treating ocular pathological conditions and/or in preventing and/or treating intestinal microbiota imbalances.

### STATE OF THE ART

Ocular pathological conditions include many eye conditions that are often accompanied by discomfort. These different disorders can occur in various forms such as stinging, itching, a burning sensation, sand or foreign body sensations in the eyes, or sensitivity to light, visual disturbance, fatigue or ocular pain, or headaches, but they can also result from tear film instability, inflammation of corneal cells, or inflammation of goblet cells in the eyes.

These symptoms may also be associated with other diseases such as ocular dryness syndrome, meibomian gland dysfunction, allergic conjunctivitis, keratoconjunctivitis, or blepharoconjunctivitis. Finally, this discomfort can be the result of surgery.

Studies have shown that there is a relationship between the microbiota and eye diseases (Baim and al. Experimental Biology and Medicine, 2019, 244, 419-429), and that the administration of prebiotics can reduce eye disorders (Flanagan and al., Antibiotics, 2019, 8, 1-23).

Prebiotics, dietary compounds in the form of sugars such as oligosaccharides and polysaccharides with short carbon chains are essential for the development and growth of bacteria in the intestinal flora. The breakdown products of prebiotics are released into the circulation and act on various organs which may be distant from each other.

One of the well-known prebiotics is inulin. This soluble fiber composed of fructose molecules is widely used as an ingredient and/or as an active compound in the food industry, for example as a supplement in infant milk powders to promote the growth of newborns with other supplements such as polyunsaturated fatty acids (Gold Mamil Growing Up Formula Step 3 from Mintel) but also in the pharmaceutical field (Hinrichs and al., Carbohydrate Polymers, 2015, 134, 418-428) and in particular for the prevention and treatment of allergies in children (WO 2013/062402), for the treatment of inflammation in combination with xylans (WO 2011/020853) or for the prevention or treatment of pathological conditions associated with the lifestyle of people (JP 201317441). Mixtures of plants, including roots known to contain inulin are also described for treatments derived from traditional Chinese medicine (CN 1245187).

Numerous pharmaceutical and nutraceutical compositions exist for the treatment of ocular diseases. However, although these compositions can act on ocular disorders, they do not allow to treat all the associated symptoms, and in particular have no action on the inflammation responsible for the discomfort.

The inventors have therefore developed a nutraceutical composition based on inulin having beneficial effects on the entire system in the case of ocular pathological conditions.

In particular, the inventors have developed a nutraceutical composition based on inulin and Chilean wineberry extract, having beneficial effects on the intestinal microbiota of people, in particular on its metabolic activity and/or on the membrane permeability of the intestines for the molecules involved in the phenomena of inflammation and/or on the composition of the microbiota, its diversity and its balance in certain microbial groups, good health.

The composition can be used preventively and/or in the context of a treatment.

### DISCLOSURE OF THE INVENTION

According to a first embodiment, the invention relates to a nutraceutical composition comprising inulin and a Chilean wineberry extract comprising anthocyanidins.

According to another embodiment, the invention relates to a formulation adapted for oral administration comprising inulin, a Chilean wineberry extract, one or more amino acids, preferably including carnitine, vitamins and minerals.

According to another embodiment, the present invention relates to a nutraceutical composition comprising inulin and a Chilean wineberry extract comprising anthocyanidins preventing and/or treating ocular pathological conditions.

According to another embodiment, the present relates to a nutraceutical composition comprising inulin and a Chilean wineberry extract comprising anthocyanidins preventing and/or treating an imbalance of the intestinal microbiota and of the pathological conditions associated with this imbalance.

### DETAILED DESCRIPTION OF THE INVENTION

### Definitions

The term “nutraceutical” refers to an edible product having a physiological beneficial effect. Thus, nutraceutical composition means a composition whose purpose is to supplement the normal diet and constituting a concentrated source of nutrients or other substances having a nutritional or physiological effect, alone or in combination.

The term “ocular pathological conditions” includes various diseases affecting the eyes. In some cases, they result in simple ocular discomfort, but can sometimes become disabling. “Ocular pathological conditions” thus means stinging, itching, burning sensations, sand or foreign body sensations in the eyes, sensitivity to light, visual disturbance, ocular fatigue, ocular pain, tear film instability, inflammation of corneal cells, inflammation of goblet cells in the eyes and/or headache. The ocular pathological conditions can also be chosen from ocular dryness syndrome, meibomian dysfunction, allergic conjunctivitis, keratoconjunctivitis, blepharoconjunctivitis, and/or be associated with eye surgery.

“Intestinal microbiota imbalance” means an imbalance in the activity of the microbial community and/or an imbalance in the microbial composition and/or an increase in the intestinal membrane permeability.

The “microbial community” is made up of all the microorganisms that make up the intestinal microbiota of an individual. Its composition, the diversity of the microbial groups present or the relative abundance of certain microbial groups are markers of an imbalance that impacts the state of health of an individual and in particular his sensitivity or his propensity to develop ocular pathological conditions. High diversity is a sign of good intestinal health.

An improvement of an imbalance of the activity of the microbial community will be observed in particular on the capacity of this community to release short-chain fatty acid, on the modulation of the concentration of lactate, on the pH, on the reduction of the amount of ammonium released and/or on gas production.

Short-chain fatty acids (acetate, propionate, butyrate, isobutyrate, isovalerate and isocaproate) are derived from the intestinal bacterial fermentation of indigestible foods, and are the main source of energy for colon cells, making them essential for gastrointestinal health.

The intestine is home to both lactate-producing bacteria and bacteria that use it. Lactate is produced by lactic acid bacteria and lowers the pH of the environment, also acting as an antimicrobial agent. Lactate can also be rapidly converted into acetate, butyrate and propionate by other microorganisms.

The measurement of pH and the degree of acidification is a measurement of the intensity of bacterial metabolism.

Ammonium is a proteolytic degradation product that results in the production of potentially toxic or carcinogenic compounds such as p-cresol and p-phenol.

Excess gas production is considered a potentially negative effect of increased saccharolytic activity of the intestinal community.

The increase in membrane permeability will result from an imbalance in the composition of the intestinal microbiota (dysbiosis) and leads to an increase in inflammation of the digestive system and to a general inflammatory state which will promote the development of pathological conditions associated with the inflammation and in particular ocular pathological conditions.

The improvement in membrane permeability is the consequence of an improvement in the composition of the intestinal microbiota which, in particular, promotes the prevention and/or treatment of ocular pathological conditions.

Other markers of intestinal microbiota imbalance are the inflammation markers pro-inflammatory cytokines (human IL-1β, IL-6, IL-8, TNF-α, CXCL10 and MCP-1) as well as the (pro-inflammatory) NF-κB activity.

A decrease in the production of these markers by the membrane epithelial cells of the intestine is the consequence of an improvement in the composition of the intestinal microbiota which, in particular, promotes the prevention and/or treatment of ocular pathological conditions.

The improvement in the imbalance of the intestinal microbiota can be quantified by the production of IL-10 (anti-inflammatory). An increase in the production of IL-10 is the consequence of an improvement in the composition of the intestinal microbiota which, in particular, promotes the prevention and/or treatment of ocular pathological conditions.

Nutraceutical Composition

The composition comprises prebiotics, natural fibers present in many plants such as fruits, vegetables, cereals and legumes, and more particularly inulins.

Inulins or inulin (CAS [9005-80-5]), are soluble dietary fibers consisting of fructose units with a terminal glucose. These natural or synthetic polysaccharides have an average degree of polymerization comprised between 2 and 60. Inulin is naturally present in many plants, such as artichokes, asparagus, bananas, leeks, Jerusalem artichokes, onions, tomatoes, barley and rye or else chicory roots, dandelion roots, and elecampane roots.

In the context of the present invention, the inulin is of natural origin, and preferentially extracted from chicory roots, dandelions and Jerusalem artichokes. In a preferred embodiment, inulin is extracted from the chicory root.

The inulin is present in an amount comprised between 1 000 and 10 000 mg, preferably between 2 000 and 8 000 mg. In one embodiment, the amount of inulin is 3 000 to 7 000 mg, preferably 4 000 to 6 000 mg, even more preferably 4 500 to 5 500 mg. In a preferred embodiment, the amount of inulin is about 5 000 mg.

In another embodiment, the inulin represents at least 50% by weight of the total composition, preferably at least 60%, and more preferably at least 70%. In one embodiment, the composition thus comprises between 70 and 90% by weight of inulin, more preferably between 80 and 90%. In a preferred embodiment, the amount of inulin represents between 82 and 89% by weight of the total composition, preferentially between 85 and 89%, and more preferentially between 86 and 88%. In a preferred embodiment, the composition comprises about 87% inulin by weight.

The composition additionally comprises a Chilean wineberry extract. This exotic fruit, also known as Aristotelia chilensis, has been used for several years in the pharmaceutical field. Several studies have shown that this natural antioxidant rich in anthocyanins and anthocyanidins has beneficial effects on the body. It can thus be used for example for the treatment of diabetes and metabolic syndrome (WO 2009/059218).

The molecules of interest present in Chilean wineberry are in particular anthocyanidins, also called anthocyanidins. In hydroxylated form, these compounds belonging to the subclass of flavonoids, exist mainly in 6 forms: cyanidol, delphinidol, pelargonidol, malvidol, peonidol and petunidol. Among these different forms, delphinidol or delphinidin is an organic compound present in many plants with strong natural antioxidant activity.

In the context of the present invention, the nutraceutical composition comprises, in addition to inulin, anthocyanidins from the Chilean wineberry. Chilean wineberry extracts are known and used pure or in high concentrations in in vitro studies (“Maqui berry extract”, from the company Oryza Oil & Fat Chemical Co.) or as active ingredients in pharmaceutical compositions or food supplements (ES 2685324; “Double Chocolate All-in-One Nutrition Bar” from the company Mintel).

Chilean wineberry extract is present in an amount comprised between 30 and 150 mg, preferably between 50 and 100 mg, more preferably between 50 and 75 mg. In another embodiment, the composition comprises between 55 and 70 mg, preferably between 55 and 65 mg of Chilean wineberry extract. In a preferred embodiment, the composition comprises about 60 mg of Chilean wineberry extract.

In one embodiment, the amount of Chilean wineberry extract represents between 0.5 and 5% by weight of the total composition. In one embodiment, the composition comprises between 0.5 and 3% by weight of Chilean wineberry extract, more preferably between 0.5 and 2%. In a preferred embodiment, the amount of Chilean wineberry extract represents between 0.6 and 1.5% by weight of the total composition, preferentially between 0.8 and 1.2%, more preferentially between 0.9 and 1.1%. In one embodiment, the composition comprises approximately 1.05% Chilean wineberry extract by weight.

The Chilean wineberry extract in particular comprises anthocyanidins and delphinidins. These compounds present in the composition represent at least 35% by weight and at least 25% by weight of the Chilean wineberry extract.

According to a particular embodiment, the inulin/Chilean wineberry extract weight ratio is at least 5, in particular at least 35, more particularly at least 50, advantageously at least 60, in particular at least 80.

According to another particular embodiment, the inulin/Chilean wineberry extract weight ratio is at most 350, in particular at most 180, more particularly at most 140, advantageously at most 110, in particular at most 90.

According to another particular embodiment, the inulin/Chilean wineberry extract weight ratio ranges from 5 to 350, in particular from 35 to 180, more particularly from 50 to 140, advantageously from 60 to 110, in particular from 80 to 90.

According to a more particular embodiment, the inulin/Chilean wineberry extract weight ratio is 82, 83, 84, 85, or 86.

In addition to inulin and Chilean wineberry, other ingredients may enter into the composition of the invention. These ingredients are well known to the person skilled in the art. It is understood that the composition according to the invention does not comprise toxic substances, or at least substances in toxic doses.

Among the compounds that can enter into the nutraceutical composition, mention will be made in particular of amino acids, which can exist in a free form or in the form of salts. Just as their stereochemistry can be of R or S configuration or of L or D type according to the plane of polarization of the light. The amino acids can thus be used in racemic or optically active forms.

In the context of the present invention, mention will be made of carnitine as the amino acid of choice. This amino acid synthesized from lysine and methionine has osmoprotective and antioxidant properties. In one embodiment of the invention, the carnitine is in the form of the R enantiomer. Thus L-carnitine will preferably be used in the formulation of the nutraceutical composition.

When the amino acids are in the form of salts, it is understood that these salts are acceptable salts for their use within the scope of the present invention. These are conventional non-toxic salts, and will obviously be apparent to the person skilled in the art. Examples of amino acid salts are tartrate, citrate, phosphate, borate, lactate, fumarate and oxalate.

In the context of the present invention, the nutraceutical composition comprises, in addition to inulin and a Chilean wineberry extract, carnitine, and more preferably L-carnitine. In one embodiment, the L-carnitine is in the form of a tartrate salt.

In the formulation, the L-carnitine in the form of the tartrate salt is present in an amount comprised between 100 and 500 mg, preferentially between 150 and 400 mg, more preferentially between 200 and 350 mg. In one embodiment, the amount of L-carnitine tartrate is from 220 to 330 mg, preferably from 240 to 320 mg, it is more preferably comprised between 270 and 320 mg. In another embodiment, the amount of L-carnitine in the tartrate salt form is comprised between 280 and 310 mg, preferably between 290 and 305 mg. In a preferred embodiment, the amount of L-carnitine in the tartrate salt form is comprised between 295 and 302 mg. It is preferably about 298 mg.

In another embodiment, the L-carnitine tartrate represents between 3 and 8% by weight of the total composition. In one embodiment, the composition thus comprises between 4 and 7% by weight of L-carnitine tartrate, more preferentially between 4.5 and 6%. In a preferred embodiment, the amount of L-carnitine tartrate represents between 5 and 5.5% by weight of the total composition, preferably between 5.1 and 5.3%. In a preferred embodiment, the composition comprises about 5.2% by weight of carnitine tartrate.

Other ingredients such as vitamins and minerals can be added to the nutraceutical composition of the present invention.

These vitamins comprise vitamins of group B, that is to say vitamin B1 or thiamine, vitamin B2 or riboflavin, vitamin B3 or vitamin PP also called niacin, vitamin B5 or pantothenic acid, vitamin B6 or pyridoxine, vitamin B8 or biotin, vitamin B9 or folic acid and vitamin B12 or cobalamin, vitamin C or ascorbic acid, vitamin D, vitamin E and vitamin K.

Advantageously, the vitamins of the present invention are vitamins A, E, C, D and vitamins of group B.

In a preferred embodiment, the composition comprises vitamins of group B, and preferably vitamin B2 or riboflavin. In another embodiment, the composition comprises vitamins of group D, and preferably vitamin D3. In another embodiment, the composition according to the invention comprises vitamins of group B and vitamins of group D, preferably vitamins B2 and D3.

In the formulation, vitamin B2 is present in the form of riboflavin in an amount greater than 0.2 mg, preferably greater than 0.6 mg, more preferably greater than 0.8 mg. In one embodiment, the amount of riboflavin is less than 2 mg. In one embodiment, the amount of riboflavin is comprised between 0.9 and 1.9 mg, preferably between 1.3 and 1.9 mg, more preferably between 1.5 and 1.8 mg. In another embodiment, the amount of riboflavin is comprised between 1.7 and 1.8 mg, preferably between 1.72 and 1.79 mg, more preferably between 1.75 and 1.78 mg. In a preferred embodiment, the amount of riboflavin is about 1.76 mg.

According to another embodiment, riboflavin represents less than 1% by weight of the total composition, preferably less than 0.5%, more preferably less than 0.2%. In one embodiment, the composition thus comprises between 0.01 and 0.2% by weight of riboflavin, preferentially between 0.015 and 0.1%, more preferentially between 0.015 and 0.05%. In a preferred embodiment, the amount of riboflavin represents between 0.02 and 0.04% by weight of the total composition, preferably between 0.025 and 0.035%. In another preferred embodiment, the composition comprises about 0.03% by weight of riboflavin, a supply form of vitamin B2.

The formulation may also comprise vitamin D, preferably vitamin D2, also called ergocalciferol (CAS [50-14-6]), and/or vitamin D3, also called cholecalciferol [CAS 67-97-0].

In one embodiment, the composition according to the invention comprises vitamin D2. In another embodiment, the composition comprises vitamin D3. In another embodiment, the composition comprises vitamin D2 and vitamin D3.

Advantageously, according to the invention, the vitamin D is vitamin D3.

When the composition comprises vitamin D in the form of vitamin D3, the latter is present in an amount greater than 0.01 μg, preferably in an amount greater than 0.1 μg, even more preferably in an amount greater than 0.5 μg. In one embodiment, the amount of vitamin D3 is less than 20 μg, or even less than 16 μg, or even less than 13 μg. In one embodiment, the amount of vitamin D3 is comprised between 1 and 12 μg, preferentially between 2 and 12 μg, more preferentially between 5 and 12 μg. In another embodiment, vitamin D3 is present in an amount between 8 and 11 μg. Advantageously, the composition comprises 9 μg of vitamin D3, preferably 10 μg.

Vitamin D3 is provided in the composition in the form of cholecalciferol. The amount of cholecalciferol according to the composition of the invention is comprised between 0.1 and 30 mg, preferentially between 0.5 and 20 mg, more preferentially between 1 and 15 mg. In another embodiment, the amount of cholecalciferol is less than 15 mg. In a preferred embodiment, the amount is thus comprised between 1 and 10 mg, preferentially between 3 and 7 mg, more preferentially between 4 and 6 mg. In a preferred embodiment, the amount of cholecalciferol is about 5 mg.

According to another embodiment, the amount of cholecalciferol represents less than 2.5% by weight of the total composition. In one embodiment, the composition thus comprises between 0.001 and 2% by weight of cholecalciferol, preferentially between 0.01 and 1%, more preferentially between 0.02 and 0.5%. In a preferred embodiment, the amount of cholecalciferol represents between 0.05 and 0.2% by weight of the total composition, preferably between 0.05 and 0.1%. Advantageously, the amount of cholecalciferol is less than or equal to 0.1% by weight, preferably less than or equal to 0.09%. In another embodiment, the amount of cholecalciferol is greater than or equal to 0.06% by weight, preferably greater than or equal to 0.07%. In another embodiment, the amount of cholecalciferol is thus comprised between 0.07% and 0.09% by weight relative to the total weight of the composition. Advantageously, the composition comprises approximately 0.087% by weight of cholecalciferol.

The minerals selected in the context of the present invention are zinc, iron, copper, selenium, manganese and chromium. These minerals can exist in a free form or in a complexed form.

Examples of zinc complexes are zinc oxide, zinc gluconate, zinc acetate, zinc citrate, zinc chloride, zinc lactate, zinc sulphate, zinc picolinate or else zinc bisglycinate. They can thus be used alone or in combination in the compositions according to the invention. Zinc bisglycinate will preferably be used in the context of the nutraceutical composition.

In the formulation, the zinc is present in an amount greater than 1 mg, greater than or equal to 2 mg, greater than or equal to 3 mg, greater than or equal to 4 mg. In one embodiment, the amount of zinc is less than 16 mg. In another embodiment, the amount of zinc is comprised between 10 and 16 mg, preferentially between 12 and 16 mg, more preferentially between 14 and 16 mg. In one embodiment, the amount of zinc is 15 mg.

In the composition according to the invention, the zinc is in a complexed form, in particular in the form of zinc bisglycinate. According to one embodiment, the amount of zinc bisglycinate is comprised between 5 and 60 mg, preferentially between 20 and 60 mg, more preferentially between 40 and 60 mg. In another embodiment, the amount of zinc bisglycinate is less than 60 mg. In a preferred embodiment, the amount is thus comprised between 50 and 59 mg, preferentially between 52 and 58 mg, more preferentially between 55 and 57 mg. In a preferred embodiment, the amount of zinc bisglycinate is about 56 mg.

In one embodiment, the zinc bisglycinate represents less than 2% by weight of the total composition. In one embodiment, the composition thus comprises between 0.1 and 2% by weight of zinc bisglycinate, preferentially between 0.3 and 1.5%, more preferentially between 0.6 and 1.2%. In a preferred embodiment, the amount of zinc bisglycinate represents between 0.8 and 1.1% by weight of the total composition, preferentially between 0.9 and 1.05%, more preferentially between 0.95 and 1.0%. In a preferred embodiment, the composition comprises about 0.98% zinc bisglycinate by weight.

Thus according to the invention, the nutraceutical composition comprises the following ingredients: inulin, a Chilean wineberry extract comprising anthocyanidins and/or delphinidins, carnitine, vitamin B12, and zinc. In a preferred embodiment, the carnitine is in the enantiomer pure form L and in the form of a tartrate salt. In another embodiment, the zinc is in the form of zinc bisglycinate complex. Finally, in another embodiment, the composition comprises carnitine in the L form and in the form of a tartrate salt and zinc in the form of zinc bisglycinate.

The nutraceutical composition according to the invention comprising 1 000 to 10 000 mg of inulin, from 30 to 150 mg of Chilean wineberry extract, from 100 to 500 mg of L-carnitine tartrate, from 0.15 to 5.0 mg of vitamin B2 or riboflavin, and 5 to 56 mg of zinc bisglycinate, is particularly suitable preventing and/or treating ocular pathological conditions.

Additives and Excipients

The nutraceutical composition can also comprise any additive allowing to improve its preservation, taste, formulation and appearance. Thus preserving, flavoring, coloring agents and formulating agents can be added. A person skilled in the art will know how to choose these agents as well as the doses necessary for the formulation.

The nutraceutical composition of the invention may additionally comprise at least one excipient and/or at least one pharmaceutically acceptable carrier.

The excipient and the carrier are “pharmaceutically acceptable” in the sense that they are compatible with the other ingredients of the composition and are non-toxic. Their use allows in particular to facilitate the preparation, storage and administration of the active compound. Such excipients and carriers are well known to the person skilled in the art, described in particular in the French or European pharmacopoeia.

Pharmaceutically acceptable excipients and carriers comprise all solvents, dispersing media, coatings, antibacterial and antifungal agents, isotonic agents, absorbents, and the like that are physiologically compatible. The excipients also comprise pH-correcting additives such as anhydrous citric acid, flavoring agents or flavors, such as for example natural red fruit flavors, and, where appropriate, sweeteners such as sucralose or stevia.

According to a particular embodiment, the composition according to the invention comprising from 1 000 to 10 000 mg of inulin and from 30 to 150 mg of Chilean wineberry extract and is formulated in a solid form.

In one embodiment, the solid nutraceutical composition comprises:
1 000 to 10 000 mg of inulin,
30 to 150 mg of Chilean wineberry extract,
100 to 500 mg of L-carnitine tartrate,
0.15 to 5.0 mg of riboflavin (vitamin B2),
5 to 60 mg of zinc bisglycinate, and
0.1 to 30 mg of cholecalciferol (vitamin D3).

In another embodiment, the nutraceutical composition comprises:
3 000 to 7 000 mg of inulin,
40 to 80 mg of Chilean wineberry extract,
150 to 400 mg of L-carnitine tartrate,
0.5 to 3.0 mg of riboflavin (vitamin B2),
30 to 56 mg of zinc bisglycinate, and
   0.5 to 20 mg of cholecalciferol (vitamin D3).

In another embodiment, the nutraceutical composition comprises:
4 000 to 6 000 mg of inulin,
45 to 75 mg of Chilean wineberry extract,
200 to 400 mg of L-carnitine tartrate,
1.0 to 2.30 mg of riboflavin (vitamin B2),
40 to 56 mg of zinc bisglycinate, and
1 to 10 mg of cholecalciferol (vitamin D3).

In another embodiment, the nutraceutical composition comprises:
4 500 to 5 500 mg of inulin,
50 to 70 mg of Chilean wineberry extract,
250 to 350 mg of L-carnitine tartrate,
1.50 to 1.90 mg of riboflavin (vitamin B2),
50 to 56 mg of zinc bisglycinate, and
3 to 7 mg of cholecalciferol (vitamin D3).

In a preferred embodiment, the nutraceutical composition comprises:
5 000 mg of inulin,
60 mg of Chilean wineberry extract,
298 mg of L-carnitine tartrate,
1.76 mg of riboflavin (vitamin B2)
56 mg of zinc bisglycinate.
4 to 6 mg of cholecalciferol (vitamin D3)

In another preferred embodiment, the nutraceutical composition comprises:
5000 mg of inulin,
60 mg of Chilean wineberry extract,
298 mg of L-carnitine tartrate,
1.76 mg of riboflavin (vitamin B2),
56 mg of zinc bisglycinate, and
5 mg of cholecalciferol (vitamin D3).

Galenic Form

Regarding the galenic formulation, several forms can be considered such as chewing gum, chewable, swallowable or effervescent tablets, gelcaps, lozenges, pills, granules, powders, drinkable solutions or suspensions. The composition according to the invention is advantageously in a solid form.

In a preferred embodiment, the composition of the present invention is in the form of a powder, tablet, gelcap or capsule.

Advantageously, the powder is dissolved forming a solution or an emulsion. The powder can also be in the form of a tablet which will be dissolved or swallowed.

Thus, in one embodiment, the nutraceutical composition is formulated for oral administration.

Dosage and Administration

According to one embodiment, the invention relates to a nutraceutical composition comprising inulin and a Chilean wineberry extract comprising anthocyanidins as defined previously for the use thereof in preventing and/or treating an imbalance of the intestinal microbiota.

According to another embodiment, the invention relates to a nutraceutical composition comprising inulin and a Chilean wineberry extract comprising anthocyanidins as defined above for the use thereof in preventing and/or treating ocular pathological conditions.

The invention relates to a method preventing and/or treating an imbalance of the intestinal microbiota in a patient in need thereof, said method comprising the administration to said patient of an appropriate amount of nutraceutical composition comprising inulin and a Chilean wineberry extract comprising anthocyanidins as defined above.

The invention also relates to a method preventing and/or treating ocular pathological conditions in a patient who needs it, said method comprising the administration to said patient of an appropriate amount of nutraceutical composition comprising inulin and a Chilean wineberry extract comprising anthocyanidins as defined above.

The composition of the present invention is formulated to be administered every day, that is to say on a daily basis. The composition is thus formulated to be administered one to four times a day. Advantageously, the composition is formulated to be administered once a day. The dosage and frequency of administration depends on several factors, including the symptoms, the severity of the pathological condition, the extent of the disorder and the physical condition of the person to be treated. A person skilled in the art will know how to adapt the dosage to be administered. It is understood that the dosage may be increased or decreased according to the physician's assessment. In one embodiment, the composition is administered three times a day. In another embodiment, the composition is administered twice a day. Advantageously, it is administered once a day.

The inulin is advantageously present in an amount (daily dose) greater than or equal to 1000 mg. According to one embodiment, the daily amount is greater than or equal to 2000 mg, greater than or equal to 3000 mg, or even greater than or equal to 4000 mg. Advantageously, the amount (daily dose) of inulin is approximately equal to 5000 mg.

Chilean wineberry extract is advantageously present in an amount (daily dose) greater than or equal to 30 mg, greater than or equal to 40 mg, or greater than or equal to 50 mg. In another embodiment, the daily amount is less than or equal to 150 mg, less than or equal to 100 mg, less than or equal to 70, preferably equal to about 60 mg.

The L-carnitine tartrate is advantageously present in an amount (daily dose) greater than or equal to 200 mg. According to one embodiment, the daily amount is greater than or equal to 250 mg, greater than or equal to 260 mg, or even greater than or equal to 270 mg.

Vitamin B2 in the form of riboflavin is advantageously present in an amount (daily dose) greater than or equal to 0.10 mg. According to one embodiment, the daily amount is greater than or equal to 0.20 mg, greater than or equal to 0.50 mg, or even greater than or equal to 0.80 mg. Advantageously, the amount (daily dose) of vitamin B2 is less than or equal to 5.0 mg, less than or equal to 4.5 mg, less than or equal to 3.5 mg. According to a preferred embodiment, the amount (daily dose) of riboflavin is approximately 1.76 mg.

The zinc bisglycinate is advantageously present in an amount (daily dose) greater than or equal to 5 mg. According to one embodiment, the daily amount is greater than or equal to 20 mg, greater than or equal to 40 mg. Advantageously, the amount (daily dose) of zinc bisglycinate is less than 60 mg, preferably less than 58 mg, more preferably less than or equal to 56 mg. According to a preferred embodiment, the amount (daily dose) of zinc bisglycinate is about 56 mg.

According to a particular composition embodiment according to the invention, the amount of cholecalciferol (form of supply of vitamin D3) is comprised between 0.1 and 30 mg, preferentially between 0.5 and 20 mg, more preferentially between 1 and 15 mg. In another embodiment, the amount of cholecalciferol is less than 15 mg. In a preferred embodiment, the amount is thus comprised between 1 and 10 mg, preferentially between 3 and 7 mg, more preferentially between 4 and 6 mg. In a preferred embodiment, the amount of cholecalciferol is about 5 mg.

### EXAMPLES

### Example 1

A nutraceutical composition is formulated as a powder to be diluted in a large glass of water and comprises 5 000 mg of inulin, 60 mg of Chilean wineberry extract, 298 mg of L-carnitine tartrate, 1.76 mg of riboflavin (vitamin B2), 56 mg of zinc bisglycinate, and 5 mg of cholecalciferol (vitamin D3).

Patients with ocular pathological conditions (stinging, itching, burning sensations, foreign body sensations, sensitivity to light, visual disturbance, eye fatigue, ocular pain, tear film instability, inflammation of corneal cells, inflammation of goblet cells in the eyes and/or headaches) are selected to follow a treatment with the nutraceutical composition for at least 30 days at the rate of one intake per day.

The patients are questioned before and after taking the nutraceutical composition, at regular intervals, to monitor their response to the treatment, the evolution of their pathological condition and any side effects.

A beneficial effect is observed for the majority of patients.

### Example 2

The effect of the composition of example 1 on the intestinal microbiota is analyzed in vitro on microbiota samples from healthy individuals. The collected cells are suspended in an appropriate medium for their conservation. 4 samples are made (1) control, (2) with addition of inulin, (3) with addition of a Chilean wineberry extract and (4) with addition of the composition of example 1.

Microbial community composition and activity analyzes are carried out and show improvement for the sample (4).

### Example 3

Analyzes of membrane permeability of the intestinal epithelial cells associated with immune parameters are carried out, (1) control without other addition, (2) with addition of inulin, (3) with addition of a Chilean wineberry extract and (4) with addition of the composition of Example 1.

Membrane permeability is measured using transepithelial electrical resistance in an in vitro model of intestinal microbiota imbalance. A drop in transepithelial electrical resistance is an indication of disruption of the intestinal epithelial cell monolayer. If the product to be tested avoids this drop, then it is capable of protecting the cells of the intestinal epithelium against hyperpermeability related to dysbiosis.

The immune parameters are measured by the production of pro-inflammatory cytokines (human IL-1β, IL-6, IL-8, TNF-α, CXCL10 and MCP-1) as well as the (pro-inflammatory) NF-κB activity. The production of (anti-inflammatory) IL10 will also be measured.

An improvement is observed for the composition of example 1.

### REFERENCES

Baim and al. Experimental Biology and Medicine, 2019, 244, 419-429
Flanagan and al., Antibiotics, 2019, 8, 1-23
Hinrichs and al., Carbohydrate Polymers, 2015, 134, 418-428
WO 2009/059218, WO 2011/020853, WO 2013/062402
JP 201317441
CN 1245187
ES 2685324

1. A nutraceutical composition comprising inulin and a Chilean wineberry extract, said Chilean wineberry extract comprising anthrocyanidins.

2. The composition according to claim 1, wherein the composition comprises 1 to 10 g of inulin and from 30 to 150 mg of Chilean wineberry extract.

3. The composition according to claim 1, wherein the inulin/Chilean wineberry extract weight ratio ranges from 5 to 350, in particular from 35 to 180.

4. The composition according to ene of claim 1, wherein the inulin/Chilean wineberry extract weight ratio ranges from 80 to 90.

5. The composition according to claim 1, wherein the inulin is extracted from the chicory root.

6. The composition according to ene of claim 1, wherein the composition further comprises cholecalciferol.

7. The composition according to claim 6, wherein the composition comprises from 0.1 to 30 mg of cholecalciferol.

8. The composition according to claim 1, wherein the composition further comprises carnitine, riboflavin and zinc.

9. The composition according to claim 1, wherein the composition comprises from 1 to 10 g of inulin, from 30 to 150 mg of Chilean wineberry extract, from 100 to 500 mg of L-carnitine tartrate, from 0.15 to 5 mg riboflavin, from 5 to 56 mg zinc bisglycinate, and from 0.1 to 30 mg cholecalciferol.

10. The composition according to claim 1, wherein the composition additionally comprises at least one excipient and/or at least one pharmaceutically acceptable carrier.

11. The composition according to claim 1, wherein the composition is in a solid form.

12. The composition according to claim 11, wherein the composition is in the form of a powder in sachet for a drinkable solution or suspension, a tablet, a gelcap or a capsule.

13. The composition according to claim 12, wherein the composition is adapted for oral administration.

14. A method for preventing and/or treating ocular pathological conditions comprising administering the composition according to claim 1 to a subject in need thereof.

15. The method according to claim 14, wherein the ocular pathological conditions comprise: stinging, itching, burning sensations, sand or foreign body sensations in the eyes, sensitivity to light, visual disturbance, ocular fatigue, ocular pain, tear film instability, inflammation of corneal cells, inflammation of goblet cells in the eyes and/or headache.

16. The method according to claim 14, wherein the ocular pathological conditions are associated with ocular dryness syndrome, meibomian dysfunction, allergic conjunctivitis, keratoconjunctivitis, blepharoconjunctivitis, and/or eye surgery.

17. A method for preventing and/or treating an imbalance of the intestinal microbiota comprising administering the composition according to claim 1 to a subject in need thereof.

18. The method according to claim 14, wherein the composition is administered once a day.

19. The method according to claim 17, wherein the composition is administered once a day.

20. The composition according to claim 1, wherein the inulin/Chilean wineberry extract weight ratio ranges from 35 to 180.
